(19) United States
(12) Patent Application Publication
(10) Pub. No.: US 2006/0252149 A1
(43) Pub. Date: Nov. 9, 2006

(54) CNS CELLS IN VITRO

(75) Inventor: Francis C. Zeigler, Encinitas, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Orion Biosolutions, Inc., Vista, CA (US)

(21) Appl. No.: 11/381,913

(22) Filed: May 5, 2006

Related U.S. Application Data
(60) Provisional application No. 60/678,608, filed on May 6, 2005.

(51) Int. Cl. C12N 5/08

(52) U.S. Cl. 435/368

(57) ABSTRACT

The invention relates to cells of the central nervous system (CNS) maintained in the presence of soluble laminin, and optionally one or more laminin associated factors, outside the CNS within an organism. The cells may be cultured in vitro or ex vivo by growth in a medium containing soluble laminin and optionally one or more of its associated factors. The invention also provides compositions comprising such cells as well as methods for their maintenance and differentiation. Additional methods of using such cells in research and therapy are also provided.

### RELATED APPLICATIONS

This application claims benefit of priority from U.S. Provisional Patent Application 60,678,608, filed May 6, 2005, which is incorporated in its entirety as if fully set forth.

### FIELD OF THE INVENTION

This invention relates to cells of the central nervous system (CNS), including those maintained outside the CNS within an organism. The cells are maintained in vitro or ex vivo by their growth in a medium containing soluble laminin and optionally one or more of its associated factors. The invention also provides compositions comprising such cells as well as methods for their maintenance and differentiation. Additional methods of using such cells in research and therapy are also provided.

### BACKGROUND OF THE INVENTION

Cells of the CNS have been cultured as “neurospheres” of cells suspended in culture to maintain them in a state capable of further differentiation. See for example U.S. Pat. No. 5,968,829. When such cells are to be induced or allowed to differentiate, they adhere to a surface to become adherent cells. The surface for adherence is typically coated with one or more extracellular matrix (ECM) proteins or cationic polymers, such as poly-ornithine and poly-lysine, which facilitate the adhesion and/or differentiation.

Cells of the CNS are believed to include multipotent precursor cells, also known as neural stem cells. These cells are capable of proliferation and are believed to give rise to transiently dividing progenitor cells that retain the ability to eventually differentiate into the cell types of an adult brain and spinal cord. Therefore, neural stem cells have been defined as having the ability to replicate and so produce more stem cells as well as to differentiate into multiple cell types having different phenotypes. These include neurons, astrocytes and oligodendrocytes.

Isolated neural stem cells have been described from several mammalian sources, including mice, rats, pigs and humans (see WO 93/01275, WO 94/09119, WO 94/10292, WO 94/16718 and Cattaneo et al., Mol. Brain Res., 42, pp. 161-66 (1996)). Neural stem cells are typically maintained as aggregates (“neurospheres”) in suspension in a mitogen-containing, serum-free medium. The mitogen may be epidermal growth factor (EGF) or EGF plus basic fibroblast growth factor (bFGF or FGF-2). Removal of the mitogen(s) and the availability of a surface as a substrate allows the cells to differentiate into neurons, astrocytes and oligodendrocytes.

Citation of documents herein is not intended as an admission that any is pertinent prior art. All statements as to the date or representation as to the contents of documents is based on the information available to the applicant and does not constitute any admission as to the correctness of the dates or contents of the documents.

### SUMMARY OF THE INVENTION

The invention relates to cells of the central nervous system (CNS) and their maintenance outside an organism while retaining the ability to proliferate and remaining in a state of being able to differentiate. The invention is based in part on the unexpected discovery that the presence of laminin, and optionally one or more laminin associated factors (hereafter “LAFs”), in solution allows cells of the CNS to proliferate under such conditions without differentiating. Moreover, the cells grow and can be propagated (or “passaged” or “passed”) under such conditions while retaining the ability to differentiate into cells with different phenotypes. The invention may thus be advantageously applied to culture and maintain primary CNS cell isolates for further use or analysis.

Thus in a first aspect, cells of the CNS that have been maintained in the presence of soluble laminin and optionally one or more LAFs as disclosed herein. The LAFs may be soluble (referred to hereafter as “sLAFs”) where a soluble LAF may be in a soluble complex with the laminin or be separately soluble. The cells of the CNS may be any that are of interest to a skilled person, including a plurality of cells with a homogeneous cell population, or a plurality of cells with a heterogeneous cell population, as non-limiting examples. In some embodiments, and based on the ability of the invention to allow CNS cells to propagate in vitro, the cells may be “monoclonal” population in that they are derived from a single cell. Thus the invention also provides for CNS cell lines derived from a single primary cell.

The invention may be practiced with cells that have been isolated from the CNS of an animal, including a primary isolate that has not been previously cultured. Alternatively, the invention may be practiced with cells that have been previously cultured outside the animal from which they were obtained, but is most advantageously practiced with such cells that retain the ability to differentiate further. In some embodiments, the CNS cells used in the practice of the invention to start a culture are not terminally differentiated and/or not postmitotic. Such cells are capable of further differentiation and/or entry into the cell cycle. Cells in the G0 phase of the cell cycle are not actively going through the cell cycle (or are “quiescent”). Cells enter the G0 phase at the beginning of what would otherwise be the G1 phase. Postmitotic cells appear to have stopped at the G phase of the cell cycle and do not seem to continue onto the other phases. Cells that are permanently in the G0 phase are called postmitotic cells. An example of such cells are neurons that express NeuN.

In other embodiments, the cells are selected from a neural stem cell, a neural progenitor cell, a neuroglial progenitor cell, a motor neuron progenitor, an oligodendroglial progenitor cell, and any CNS-derived cell which plays a beneficial role in the regenerative response to CNS damage, inflammation, or infection. In further embodiments, the cells may be a nestin expressing neuroepithelial or neuroectodermal cell, or a radial glial cell-like neuroglial progenitor cell or a motor neuron precursor. In yet another embodiment, the cells have characteristics of a CNS cell as described herein and are derived from, or descendant from, a primate-derived primordial germ cell or a pluripotent human embryonic stem cell. Alternatively, the cells are derived from, or descendant from neuroectodermal and/or neuroendocrine cells, such as cells of the neural crest, neural tube, neural fold, neural groove, anterior neuropore, posterior neuropore, and germinal neuroepithelium, as non-limiting examples. In some embodiments, a characteristic of a CNS cell is nestin expression.

CNS cells are distinct from cells of the peripheral nervous system (PNS) and cells found in non-CNS tissues, such as in the gut. Of course the invention also includes a daughter or descendant cell obtained by passage of a CNS cell in culture with soluble laminin and at least one LAF or sLAF as provided herein.

Without being bound by theory, and offered to improve the understanding of the invention, cells that have been cultured with soluble laminin and optionally at least one LAF in accordance with the invention may have a different phenotype than cells that have not been so exposed. This is based upon a combination of observations, beginning with the culture conditions disclosed herein include use of a mitogen at levels not seen in vivo or soluble laminin, optionally with at least one LAF, which is not present in vivo. The culture conditions are not identical to the in vivo environment. Moreover, these conditions result in the cells being prevented from differentiating further, or terminally differentiating, as would be the case of culture conditions lacking a mitogen or soluble laminin (optionally with an LAF) combination. As such, even cells that are committed to differentiate do not progress further on the differentiation pathway, or otherwise display the phenotype of a further differentiated cell, when cultured or maintained in accordance with the disclosed invention. Thus it appears that the cells have a difference in expression of at least one gene product which renders them different from cells initially isolated from an in vivo environment and from differentiated cells. Stated differently, but without necessarily limiting the invention, the cells may have a new and different phenotype.

In a second aspect, the invention provides cells that are derived from a cell or cells cultured in the presence of soluble laminin and optionally at least one LAF. In many embodiments, such cells may be considered differentiated cells relative to the cells cultured with soluble laminin and optionally one or more LAFs. Generally, such a derivative cell would display, as all or part of a new phenotype, an increase in expression of at least one marker of a differentiated CNS cell type. The derivative cell can also display, as part of a new phenotype, a decreased level of nestin expression or a increased level of glial fibrillary acidic protein (GFAP) expression. In other embodiments, a derivative cell may have an increased level of TUJ1/β-tubulin expression as part of its phenotype.

In some embodiments, the derivative cell expresses one or more of a marker selected from acetyl cholinesterase; choline acetyltransferase; vesicular acetylcholinesterase; gamma-aminobutyric acid (GABA); serotonin; a synapse marker, including synaptophysin and synaptogamin; post-synaptic density protein 95 (PSD-95); myelin basic protein; myelin associated glycoprotein (MAG); proteo-lipid protein (plp or DM20); tyrosine hydroxylase; and L-3,4-dihydroxyphenylalanine (DOPA) decarboxylase; MAP2; neuron specific enolase; synapsin I. Given that these markers are not generally recognized as expressed in CNS stem and/or progenitor cells cultured with soluble laminin, and optionally at least one LAF, such derivative cells necessarily have a phenotype distinct from stem and progenitor cells in culture. In additional embodiments, the derivative cell is an astrocyte, a neuron, a dopaminergic neuron, an interneuron, a motor neuron, an oligodendrocyte, or a Schwann cell. Given the ability to propagate less differentiated cells with laminin and optionally at least one LAF as provided herein, the invention provides the ability to produce very large quantities of differentiated cells.

Induction of differentiation in the cultured cells may be by any appropriate means, including withdrawal of mitogen, soluble laminin, and/or the present LAF(s). Other non-limiting examples include induction with retinoic acid, or a neurotrophic peptide factor such as neural growth factor (NGF) and/or the neurotrophins (NT-3 and NT-4), neuregulins, glial cell derived neurotrophic factor (GDNF), ciliary neurotrophic factor (CNTF), and brain derived neurotrophic factor (BDNF).

In another aspect, the invention provides for compositions and preparations of cells either in the presence of soluble laminin and at least one LAF or cells that have been previously cultured in the presence of soluble laminin and at least one LAF. Thus the invention includes a culture of cells in media containing soluble laminin and optionally at least one LAF, per se, as described herein. The invention also includes a preparation of cells that have been cultured in the presence of soluble laminin, and optionally at least one LAF, such that the cells are distinct from previously known cells. The invention further includes a composition or preparation of cells previously cultured with soluble laminin, and optionally at least one LAF, and at least one agent which induces differentiation. Compositions and preparations of daughter, descendant, and derivative cells are also provided by the instant invention.

In a further aspect, the invention provides for culture media comprising a combination of soluble laminin and optionally at least one LAF. The invention also provides for transplantation media comprising such a combination of soluble laminin and optionally at least one LAF. The ability to use soluble laminin, and optionally one or more LAFs, to culture CNS cells as provided herein reflects an unexpected discovery because laminin is an ECM component that may be coated onto a surface used in the differentiation of CNS cells. Thus the use of a soluble form of laminin, and optionally an LAF, to maintain cells in a state without further or terminal differentiation, and with enhanced proliferation and/or expansion in culture, is a surprising discovery. The discovery is even more unexpected in light of other work describing soluble laminin as inducing differentiation of fetal mouse pancreatic precursor cells to form β-cells (see Jian et al. Diabetes. 48:722-730, 1999).

In some embodiments, the soluble form of laminin is a salt extract of basement membranes or ECM that contains laminin. A non-limiting example includes laminin prepared by the process of Timpl et al. (J. Biol. Chem., 254(19):9933-9937 (1979)). See also Timpl et al. TIBS 8:207-209 (1982). Such soluble laminin may be advantageously used in some embodiments of the invention because it already contains at least one LAF as part of a soluble laminin containing complex. Further embodiments include the preparation of laminin from the basement membranes of Engelbreth-Holm-Swarm (EHS) mouse sarcoma cells, although other cellular sources of laminin, including other murine and human cells and basement membranes, may also be used. Commercially available sources of soluble laminin may also be used in the practice of the invention.

The one or more LAF may be selected from those that are found in the presence of soluble laminin, including those that are part of a complex with laminin or those that are in free solution apart from laminin. Non-limiting examples include nidogen (also known as entactin); a heparan sulfate containing proteoglycan (such as Perlecan); collagen type IV; secreted protein, acidic, rich in cysteine (SPARC); tenascin; reelin; or thrombospondin. A combination of one or more of the above LAFs may also be used in the practice of the invention. Moreover, isoforms of laminin or an LAF may also be used, with the gamma-1 chain isoform of laminin being a non-limiting example. Given the presence of a nidogen-binding site in the gamma-1 isoform, nidogen would be readily present as an LAF with this isoform. As additional non-limiting examples, nidogen-1 (entactin-1) and/or nidogen-2 (entactin-2) may be used in combination with laminin in the practice of the invention. The use of a nidogen is facilitated by the observation that nidogen is found complexed with soluble laminin, sometimes in a molar ratio of about 1 to about 3 nidogen to one laminin molecule.

As noted above, culture media containing soluble laminin, and optionally at least one LAF, permit the proliferation of CNS cells in culture. The conditions have been observed to permit an approximate 8× to 10× increase in cell number in about 7 days. In some embodiments of the invention, the doubling time of the cells is from about 24 to about 72 hours, including about 36, about 48, and about 60 hours. This is in contrast to the 5-10 day doubling time of human CNS derived neurospheres as described in U.S. Pat. No. 6,498,018, where soluble laminin was not present. The invention thus includes cells of the CNS under conditions with a doubling time of less than 5 days, as well as those with a doubling time of about 24-72 hrs. Such cells may comprise greater than about 50% that are immunoreactive for nestin expression, up to about 75 to about 99% immunoreactive.

An additional aspect of the invention is a method of using a medium containing soluble laminin, and optionally at least one LAF, to culture and/or propagate CNS cells. The method may be used to grow and/or maintain CNS cells in culture for extended periods of time until senescence and termination of cell divisions. In some embodiments, the number of cell divisions during culture would be about 70.

In some embodiments, the methods are used to culture the cells as adherent cells. Non-limiting examples include planar and 3-dimensional formats. Non-limiting examples of 3-dimensional formats include hydrogels (including purified human collagen hydrogels) composed of basement membrane components from cells as well as growth on porous, biocompatible polymer scaffolds, such as nylon screens with pore sizes between about 70 and about 200 μm. Alternatively, the methods are used with cells grown in suspension, including “neurospheres” where they are cultured in the presence of soluble laminin and optionally at least one LAF. Cultures “in suspension” refers to those where cells are either incapable of being adherent to a surface of the culture device used, or where a deliberate means or mechanism is used to prevent cell adherence to the device used.

In further embodiments, methods for the use of cells cultured or otherwise propagated in accordance with the invention are provided. One non-limiting example is a method of determining the effects of a candidate agent on CNS cell differentiation, growth, viability, or metabolic activity by testing the activity of the agent on CNS cells under the invention's culture conditions with soluble laminin and optionally at least one LAF. Such a method may be practiced in combination with a change in culture conditions to permit differentiation such that the effects of the agent on differentiation are observed. The observations may be compared to those in a control culture not treated with the agent but otherwise identically processed. The change in culture conditions may include the withdrawal of mitogen, soluble laminin, and/or the present LAF(s) as non-limiting examples. Alternatively, the change may include the induction of differentiation as described herein.

Moreover, the invention provides methods for transplanting CNS cells propagated as described herein to a recipient host organism. The availability of larger numbers of cells as well as the retention of a state of being able to differentiate improves the viability and usefulness of the cells upon transplant. As a non-limiting example, cells cultured under the invention's conditions can be transplanted to the site of spinal cord injury to provide therapeutically-beneficial cell phenotypes, such as, but not limited to, motor neurons, motor neuron progenitors or precursors, Schwann cells, oligodendrocytes, and oligodendroglial progenitors. The cells of the disclosed invention, when transplanted into the damaged spinal cord, would accomplish the partial or complete restoration of spinal cord function. As another non-limiting example, human pluripotent embryonic stem cells or cells derived from them may be cultured under the invention's conditions, maintaining the capacity to differentiate into CNS cell types for transplantation into CNS sites where neurodegeneration or dysfunction has led to a disease state, such as Parkinson's disease, Alzheimer's disease, or Huntington's disease, or the normal loss of CNS cell function with aging, thereby providing a therapeutic benefit. It will be obvious to the person skilled in the art, that transplantation can be accomplished through a variety of modalities. Non-limiting examples of transplantation modalities for cells cultured by the disclosed invention, include intracranial and/or intrathecal injection using a syringe and large gauge needle, such as an 18 gauge needle. Further non-limiting examples of transplantation modalities for cells of the invention include implantation through incisional wounds, especially those produced through surgical procedures within the vicinity of the damaged or dysfunctional area of the CNS, by way of a biocompatible carrier, such as cell culture medium and biodegradable polymers made form polyglycolic acid and/or polylactic acid and/or purified collagen hydrogels.

In additional embodiments, a kit comprising the cells or media of the invention is provided by the invention.

The details of one or more embodiments of the invention are set forth in the accompanying drawings and the description below. Other features, objects, and advantages of the invention will be apparent from the drawings and detailed description, and from the claims.

### BRIEF DESCRIPTION OF THE DRAWINGS

FIG. 1 shows differences in overall cell expansion and proliferation rate in vitro between human neurospheres and adherent cultures with soluble laminin and nidogen complexes from mid-gestation (18 weeks) fetal brain. The growth in culture was for 10 weeks. The x-axis shows passage number ranging from P0 to P10, and days in vitro (DIV) from 0 to 70 after isolation. The y-axis is “total cell expansion” ranging from 10⁵ cells to 10¹¹ cells. Neural stem cells cultured in the presence of soluble laminin complexes maintain a stable proliferation rate during expansion in vitro, while neurosphere cultures maintained in the same medium without the use of laminin complexes do not. Also, it is shown that the neurosphere cultures initially (within the first passage after 7 DIV) display a net loss of total cells, and that their proliferation rate only stabilizes after passage 5 and 35 DIV. The same cells cultured in the presence of soluble laminin complexes do not display such effects in cell growth.

FIG. 2 shows the quantification of phenotypes from fourth passage 4E18 rat neural stem/progenitor cells cultured in the presence of soluble laminin and sLAFs. The cells were immunostained via intracellular fluorescence activated cell sorting (FACS). Panel A shows irrelevant immunoglobulin controls; Panel B shows the population of nestin and glial fibrillary acidic protein positive cells; Panel C shows the population of NeuN and myelin basic protein positive cells; and Panel D shows the population of beta-tubulin III and glial fibrillary acidic protein)

### DETAILED DESCRIPTION OF MODES OF PRACTICING THE INVENTION

The invention relates to the isolation, proliferation, differentiation and transplantation of CNS cells, but given the benefit of their growth in the presence of soluble laminin and optionally at least one LAF. The invention is based in part on the observation that soluble laminin, optionally in the presence of a laminin associated factor (LAF), allows the maintenance of CNS cells in culture with little to no increase in the number or type of further, or terminally, differentiated cells from less differentiated cells. This allows for the ability to grow CNS cells in culture for extended periods while the cells retain their ability to differentiate further under appropriate conditions.

The invention thus provides compositions containing the soluble laminin, optionally with one or more LAF, which compositions may be used with CNS cells. The invention further provides CNS cells that have been grown or cultured in the presence of soluble laminin, optionally with one or more LAF, or in the presence of a composition of the invention. Of course cells that are derived from such CNS cells are also provided. Further provided are methods of maintaining CNS cells under conditions with soluble laminin and optionally one or more LAF, such as with a composition of the invention.

In one aspect, the invention provides a composition of cells comprising a cell of the central nervous system (CNS) that has been cultured in the presence of a solution comprising laminin and optionally one or more LAF. Optionally, the LAF may be in soluble form, as a complex with the soluble laminin or separate from the laminin.

The CNS cells may be isolated from a variety of brain and spinal tissue in various animals. In some embodiments, the invention may be practiced with human CNS stem cells isolated from the forebrain. The isolated cells may be used to prepare cell lines by use of the compositions and methods of the invention. In some embodiments, the isolated cells exhibit neural stem cell properties. For example, the cells are self replicating or renewing (without progress toward a more differentiated state); proliferate for long periods in serum-free, mitogen containing medium; and the cells may be subsequently differentiated, such as to a population of neurons, astrocytes, and/or oligodendrocytes. In some embodiments, the cell is a nestin expressing neuroepithelial cell or radial glial cell-like neuroglial progenitor cell. In other embodiments, the cells are neuroectodermal and/or neuroendocrine cells, such as nestin expressing cells. Non-limiting examples of such cells include those of the neural crest, neural tube, neural fold, neural groove, anterior neuropore, posterior neuropore, and germinal neuroepithelium.

Other non-limiting examples of CNS cells as disclosed herein include cells of the mesencephalon (or midbrain); rhombencephalon (hindbrain), such as myelencephalon, medulla or medulla oblongata, metencephalon, pons, or cerebellum; proscencephalon (forebrain), such as diencephalons, telencephalon, or neopalluml; or cells derived or descendant therefrom. Additional non-limiting examples include neuroblasts, neural tube epithelial cells, spongioblasts, or cells derived or descendant therefrom.

It is appreciated that CNS cells isolated from a naturally occurring source may be a heterogenous population. Thus an isolated population of cells may include cells that express one or more cellular factors selected from nestin, glial fibrillary acidic protein, NeuN, myelin basic protein, and beta-tubulin III. Such heterogeneous cell populations may be used in some embodiments of the invention, especially given the observation that the proportion of cells in the population expressing these factors remains relatively constant under the conditions of the disclosed invention. Alternatively, the invention may be practiced with cell populations that are homogenous or less heterogeneous. Where nestin expressing cells are the predominant species in a population of cells, the proportion of nestin expressing cells is greater than about 50%, such as greater than about 60, 70, 80, or 90%. Populations with about 90 to about 95% or higher have been successfully used in the practice of the invention. Similarly, cell populations may have expression of GFAP of less than about 20%, such as from less than about 1%, less than about 2%, less than about 4%, less than about 6%, less than about 8%, or less than about 10%. The expression of β-tubulin III and MBP in the cell population may be independently less than 5%, such as less than about 1%, less than about 2%, less than about 3%, or less than about 4%, for each of the two cell factors.

A variety of suitable sources may be used to obtain CNS cells for the practice of the invention. Non-limiting examples include any tissue containing CNS cells as well as a range of different animals with a CNS, including mammals, primates(including non-human primates), aquatic species, avian species, and reptilian species. Cells from humans, mice, pigs, cattle, sheep, goats, rats, rabbits, and chimpanzees may be used in the invention. The CNS cells may also be from a member of a species at various times during development, including but not limited to, a fetal organism, a newborn organism, a young organism, a young adult organism, an adult organism, an older adult organism, and an elderly adult organism. Other non-limiting examples include CNS cells from in utero or mid-gestational tissues. And while CNS cells may be cultured in the presence of a soluble laminin and optionally at least one LAF from the same species, the invention is not so limited. Thus laminins and LAFs and the cells maintained therewith may be from different species. Non-limiting examples include where the CNS cells are human or rat in origin while the laminins and LAFs are murine in origin. The CNS cells may be obtained as allografts and autografts contemplated for subsequent transplantation purposes.

A variety of LAFs, including isoforms and species homologs, may be used individually or in combination in the practice of the invention as discussed herein. Non-limiting examples include the two forms of human nidogen (also known as entactin) referred to as nidogen-1 (or entactin-1) and nidogen-2 (or entactin-2). The former has a deposited sequence with GenBank Acc. No. NM 002508 while the latter has a deposited sequence with GenBank Acc. No. NM007361 (nidogen/entactin-2). The invention contemplates use of nidogen-1 or nidogen-2 or both with equal success. Other LAFs include heparin sulfate proteoglycan; collagen type IV; secreted protein, acidic, rich in cysteine (SPARC); tenascin; reelin; thrombospondin; or a combination of any number of the foregoing. An LAF may be separately soluble from laminin or be soluble as part of a laminin containing, soluble complex. In cases of an LAF that is a polypeptide, the invention also provides for the use of a recombinantly produced form of the LAF. For example, a recombinantly produced nidogen-1 and/or nidogen-2 may be used in the practice of embodiments comprising the use of nidogen/entactin.

Similarly, a wide variety of soluble laminins may be used in the practice of the invention. Non-limiting examples of sources include Chemicon International (mouse laminin, catalog no. CC095), Roche Applied Science (mouse laminin, catalog no. 1 243 217), and Sigma-Aldrich (mouse laminin, catalog no. L 2020). Of course a skilled person in the field can also prepare laminin from any suitable source by the method of Timpl et al. as cited above. Generally, sources of laminin are any laminin containing cellular material, such as basement membranes found with cells and cell lines. Non-limiting examples include human epithelial cells, including the HaCaT keratinocyte cell line, and primary human dermal fibroblasts in cultures, including 3D cultures. In additional embodiments, the soluble laminin is present in combination with soluble type IV collagen, which may be indirectly associated with the laminin or separately soluble in solution. Complexes of laminin and collagen may be enhanced by the inclusion of zinc ions as described by Ancsin et al. (J. Biol. Chem., 271(12):6845-6851, 1996). The combination of soluble laminin and collagen may also be present with entactin/nidogen, optionally as a complex of all three molecules.

In further embodiments, the laminin may be a recombinantly produced molecule, such as one comprising all or part of the gamma-1 chain of laminin, or a portion of laminin which retains entactin/nidogen binding capability, as non-limiting examples. A laminin molecule comprising the zinc finger-containing Cys-rich repeat on the gamma-1 chain, such as a polypeptide containing the YIGSR sequence, may also be used in the practice of the invention. Recombinant production of polypeptides, based upon the use of recombinant nucleic acid molecules that encode the desired polypeptide may be advantageously used as deemed desirable by the skilled person.

In some embodiments of the invention, CNS cells are exposed to final concentrations of soluble laminin from about 100 ng/ml to about 100 μg/ml or higher. Thus the invention may be practiced with soluble laminin of about 100 ng/ml, about 200 ng/ml, about 400 ng/ml, about 600 ng/ml, about 800 ng/ml, about 1 μg/ml, about 2 μg/ml, about 4 μg/ml, about 6 μg/ml, about 8 μg/ml, about 10 μg/ml, about 20 μg/ml, about 40 μg/ml, about 60 μg/ml, or about 80 μg/ml, or higher. Such levels of soluble laminin are higher than that of laminin which is merely liberated from laminin coated surfaces, such as a coated dish or plate. The soluble laminin may be added in whole or in part to the cells to arrive at the above levels. The range of concentrations of one or more LAFs for use in the invention is the same as that for laminin as described above. The LAF(s) may also be added in whole or in part to the cells to arrive at the concentrations.

As described herein, the soluble laminin and one or more LAFs are used with one or more mitogens in media for CNS cells. Mitogens of the invention include, but are not limited to, epidermal growth factor (EGF), EGF plus basic fibroblast growth factor (bFGF or FGF-2), leukemia inhibitory factor (LIF), and combinations thereof. The amounts of mitogens may vary depending on the nature of the cell used and as known by the skilled person in the field of cell culture.

Other compositions of the invention include those that comprise soluble laminin and one or more LAF as described above. In some embodiments, the invention provides culture or growth media for use with the CNS cells described herein. Such a medium may comprise soluble laminin as well as at least one LAF, optionally in combination with one or more mitogens as described. The medium may be any standard culture medium, including serum-free or serum-depleted media containing from 0 to about 0.5% serum.

Non-limiting examples of such media include Fischer's, alpha medium, Leibovitz's, L-15, NCTC, F-10, F-12, DMEM, MEM, McCoy's, Iscove's modified Dulbecco's medium (“IMDM”), and RPMI, optionally with one or more additives, including serum albumin, heparin, non-essential amino acids, putrescine, ITS supplement (Sigma), and mitogens, like bFGF and LIF, as non-limiting examples. In some embodiments, the media may be in concentrated form such that it must be diluted before use. In other embodiments, the media may be serum-free or serum-depleted, for short term and long term proliferation, respectively, of CNS cells. Non-limiting examples of serum-free or serum-depleted culture media are known in the field (see for example WO 95/00632).

Other cells of the invention include those that have been maintained in vitro or ex vivo in the presence of soluble laminin, optionally with one or more LAF, as described herein. The laminin and optional LAF may have been in a composition of the invention, or other solution containing laminin and optional LAF(s), as described. Such cells of the invention include CNS cells that are cryopreserved according to routine procedures known in the field. Such forms of the cells are a further composition and/or preparation of the invention. The cryopreserved form may contain medium as described herein, preferably in the presence of DMSO. The cells may be slowly frozen, and quickly thawed.

As explained, a maintained or cultured cell population includes cells that are not terminally differentiated and/or not postmitotic. In some embodiments, the population includes cells selected from neural stem cells, neural progenitor cells, motor neurons, and regenerating cells as described herein. In other embodiments, the population includes cells that are nestin expressing neuroepithelial cells and/or radial glial cell-like neuroglial progenitor cells.

The invention further provides cells that are derived from the maintained or cultured CNS cells of the invention. Such derived cells include cells maintained with soluble laminin, and optionally one or more LAF, that are subsequently differentiated into a more differentiated cell type. Derived cells also include those that have lost some or all of their capability to differentiate further. Non-limiting examples of such derived cells include those that display reduced, or the absence of, nestin positive immunoreactivity, such that the cells have reduced or no immunoreactivity with anti-nestin antibodies.

The detection of various cell surface or intracellular markers may be used to identify cellular phenotype either during proliferation or differentiation of the CNS cells of the invention. As a non-limiting example, when the CNS cells of the invention are proliferating, anti-human nestin antibody may be used as a marker to identify undifferentiated cells. Similarly, anti-β-tubulin antibodies can be used to show little, if any, β-tubulin expression in a population of cells.

In some embodiments, antibodies specific for various neuronal or glial proteins may be employed to detect markers indicative of phenotypic properties of differentiated cells. Antibodies to neuron specific enolase (“NSE”), neurofilament, tau, β-tubulin, or other known neuronal markers may be used to identify neurons. Antibodies to glial fibrillary acidic protein (“GFAP”) and other known astrocytic markers may be used to identify astrocytes. Antibodies to galactocerebroside, O4, myelin basic protein (“MBP”) and other known oligodendrocytic markers may be used to identify oligodendrocytes.

Alternatively, cell phenotypes are identified by detecting compounds characteristically produced by those phenotypes. As non-limiting examples, neurotransmitters such as acetylcholine, dopamine, epinephrine, norepinephrine, and the like are produced by neurons. Thus detection of such products produced by a cell would indicate a cell as being a neuron. By extension, specific neuronal phenotypes may be detected by the specific products produced by neurons with those phenotypes. As a non-limiting example, GABA-ergic neurons may be detected by their production of glutamic acid decarboxylase (“GAD”) or GABA. As another non-limiting example, dopaminergic neurons may be detected by their production of dopa decarboxylase (“DDC”), dopamine or tyrosine hydroxylase (“TH”). As an additional non-limiting example, cholinergic neurons may be detected by their production of choline acetyltransferase (“ChAT”). As a further non-limiting example, hippocampal neurons may be identified by detection of NeuN.

In some embodiments of the invention, the derived cells are those that display a decreased level of nestin expression, optionally with an increased level of TUJ1/β-tubulin expression; a increased level of glial fibrillary acidic protein (GFAP) expression; or an increase in expression of at least one marker of a differentiated CNS cell type. In other embodiments, a derived cell has an increased expression of a marker selected from acetyl cholinesterase; vesicular acetylcholinesterase; gamma-aminobutyric acid (GABA); serotonin; a synapse marker, including synaptophysin and synaptogamin; post-synaptic density protein 95 (PSD-95); myelin basic protein; myelin associated glycoprotein (MAG); proteo-lipid protein (plp or DM20); NG2 proteoglycan; CD24; CD133; CD49f; tyrosine hydroxylase; and/or L-3,4-dihydroxyphenylalanine (DOPA) decarboxylase. Further embodiments include a derived cell that is an astrocyte, a neuron, an oligodendrocyte, or an oligodendroglial cell.

In addition to derived cells, the invention provides for a daughter or descendant cell of any CNS or CNS derived cell of the invention. A daughter or descendant cell is one that is obtained by passage of a parental cell by use of the compositions or methods of the invention comprising soluble laminin and optionally one or more LAF. Compositions comprising a daughter or descendant cell, such as a homogeneous or heterogeneous population of such a cell, are also provided by the disclosed invention.

The aspects and embodiments of the invention are based upon methods of maintaining CNS cells under conditions with soluble laminin and optionally one or more LAF. Thus the invention also provides a method of maintaining, culturing, or otherwise growing a cell of the CNS ex vivo or in vitro. Such a method maintains, cultures, or otherwise grows said cell in a medium containing soluble laminin, optionally with one or more LAF. The contact of a cell with soluble laminin, and optional LAF(s), may be performed as early as possible, such as with the first medium used to provide nutrients to the cell, and may be maintained for as long as desired, such as in all media that the cells contact. In some embodiments, the soluble LAF is nidogen, optionally in a soluble complex in a medium used to culture the cells. The medium may be any suitable medium known to the skilled person in the field, including those described above and herein. The medium may further comprise a mitogen as described herein, such as FGF-2, EGF, PDGF, and/or NGF, optionally with LIF (leukemia inhibitory factor), as non-limiting examples. Analogs, derivatives and/or combinations of the mitogens, such as EGF and bFGF in combination, may also be used.

The methods of maintaining, culturing, or otherwise treating CNS cells with soluble laminin and optionally one or more LAF may be conducted with culture techniques and devices known in the field. Non-limiting examples include adherent cultures as well as three dimensional culture formats. With adherent cultures, the invention may be practiced with or without previous immobilization (coating) of laminin on the surfaces to which the cells adhere. In some embodiments of the invention, the medium may be optionally pre-conditioned under incubation conditions to allow temperature and pH equilibration. A non-limiting example of such pre-condition is for about 30 minutes to about 1 hour in an incubator.

Additional methods are used to determine or observe the effects of a candidate agent on CNS cell differentiation, growth, viability, or metabolic activity. Such methods comprise the presence of soluble laminin and optionally one or more LAF, where the presence is either maintained during contact with the candidate agent or removed prior to the contact. In some embodiments, a method includes testing the activity of the agent on CNS cells with soluble laminin, and optionally at least one LAF, present, where the culture conditions are then adjusted to permit differentiation of at least some of the cells of the culture. After adjustment, the effects of the agent on differentiation, growth, viability, or metabolic activity may then be observed or determined. The observations may be compared to those in a control culture not treated with the agent but otherwise identically processed. The change in culture conditions may include the withdrawal of mitogen, soluble laminin, and/or the present LAF(s) as non-limiting examples. Alternatively, the change may include the induction of differentiation as described herein.

In other embodiments, a method includes maintaining CNS cells as described herein followed by removal of the soluble laminin, and optional LAF, prior to a change in conditions to allow differentiation of the cells. The candidate agent may be introduced simultaneously with, or after, the change in conditions. Again the effects of the agent on differentiation, growth, viability, or metabolic activity may then be observed or determined, where the observations may be compared to those in a control culture not treated with the agent but otherwise identically processed.

In one embodiment, a method of determining the effects of a candidate agent on a cell of the CNS differentiation, growth, viability, or metabolic activity is provided, with the method comprising culturing said cell in a medium containing soluble laminin and soluble nidogen; altering the culture conditions for said cell to permit differentiation, such as by growth factor withdrawal, after said cell has been contacted with said candidate agent; and observing the effects of said candidate agent on said differentiation in comparison to another cell cultured under the same conditions to permit differentiation in the absence of said candidate agent. The change in culture conditions to permit differentiation may be selected from withdrawal of soluble laminin and nidogen and/or induction with retinoic acid, or introduction of a neurotrophic peptide factor such as neural growth factor (NGF), neuregulins, glial cell derived neurotrophic factor (GDNF), ciliary neurotrophic factor (CNTF), and brain derived neurotrophic factor (BDNF).

In further embodiments, the CNS cells of this invention may be directly transplanted as a graft into a subject according to conventional techniques, such as by use of parenchymal and intrathecal sites. Transplantation may be into the CNS of an animal, such as that described in U.S. Pat. Nos. 5,082,670 and 5,618,531. Alternatively, the transplantation may be into another suitable site in the body, depending on the needs of the subject as diagnosed or determined by the skilled practitioner or medical professional. The amount of cells transferred may range from about 10,000 to about 1,000,000 cells per μl.

The transplanted/implanted cells may be labeled prior to transplantation. A non-limiting examples is with use of bromodeoxyuridine (BrdU). The label may be used as part of a double staining for the label and a cell marker to show that the labeled cells differentiated into one or more particular neural cell types after transfer. Alternatively, the cells may be encapsulated or microencapsulated prior to transfer such that afterwards, the cells express and deliver biologically active molecules expressed by the cells. Such methodologies are known in the field and may be directly used or adapted for use with the disclosed invention.

The methods of the invention have resulted in differentiated neural stem cell cultures that are highly enriched in GABA-ergic neurons after transplant into an area such as the hippocampus. The origins of the cells include the human forebrain. Such GABA-ergic neuron enriched cell cultures are particularly advantageous in the potential therapy of excitotoxic neurodegenerative disorders, such as Huntington's disease or epilepsy.

Other neurodegenerative diseases and disorders may also be treated by transfer of the cells of the invention. Non-limiting examples include pathological conditions resulting from excitotoxicity, such as, but not limited to, epilepsy, stroke, ischemia, and neurodegenerative diseases like Parkinson's disease and Alzheimer's disease. The cells may be used to replace or augment the function of endogenous cells or tissue in the subject.

Cell populations of the invention that are enriched in oligodendrocytes or oligodendrocyte precursor or progenitors, may be used to promote remyelination of demyelinated areas in a subject. This may be advantageously used in the treatment of various demyelinating and dysmyelinating disorders. Non-limiting examples include Pelizaeus-Merzbacher disease, multiple sclerosis, various leukodystrophies, post-traumatic demyelination, and cerebrovascular (CVS) accidents.

The CNS cells described herein, as well as their differentiated and undifferentiated progeny, may be made immortal or conditionally immortal by known techniques. Such immortalized cells may be maintained as a cell line by methods known in the field. Conditional immortalization techniques include Tet-conditional immortalization (see WO 96/31242), and Mx-1 conditional immortalization (see WO 96/02646).

The invention further provides for articles of manufacture to maintain or culture CNS cells as described herein. An article of manufacture according to the disclosed invention may be a kit for the practice of the methods disclosed herein or an article containing one or more reagents needed to practice the methods. The kit can comprise the soluble laminin, optionally with one or more LAF as described herein, as well as optionally one or more other reagents, for use in the disclosed invention, together with suitable packaging material. Preferably, the packaging includes a label or instructions for the use of the article or kit in a method disclosed herein. A kit of the invention may also comprise any composition of the disclosed invention, optionally for use in a method described herein.

Having now generally described the invention, the same will be more readily understood through reference to the following examples which are provided by way of illustration, and are not intended to be limiting of the disclosed invention, unless specified.

### EXAMPLES

The following examples are offered to illustrate, but not to limit the claimed invention.

### Example 1

Mid-gestation human fetal brain (18 weeks) was minced briefly with two opposing scalpels, then incubated for 30 minutes in 10 ml of 1 mg/ml collagenase H (Boehringer Mannheim) in Hank's Buffered Salt Solution in a 37C water bath with occasional agitation. The digest was then diluted with Hank's by addition of approximately 40 ml, and centrifuged at 200 g for 7 minutes. The resulting cell pellet was washed once in Hank's, then resuspended in 0.05% trypsin/EDTA and incubated again in a 37° C. water bath for 10 minutes. After addition of Hank's with 0.2% serum albumin added (40 ml), and centrifugation as before, the cell pellet was resuspended in neural stem cell medium (DMEM/F12:DMEM 50:50 (v/v) with 0.1% serum albumin, 2 ug/ml heparin, 1× non-essential amino acids, 10 ng/ml putrescine, 1× ITS supplement (Sigma), 10 ng/ml bFGF, and 5 ng/ml LIF (both from Sigma). Viable cell counts were determined with a hemacytometer using trypan blue, and 1×10⁶ viable cells were plated in duplicate 150 cm² T-flasks.

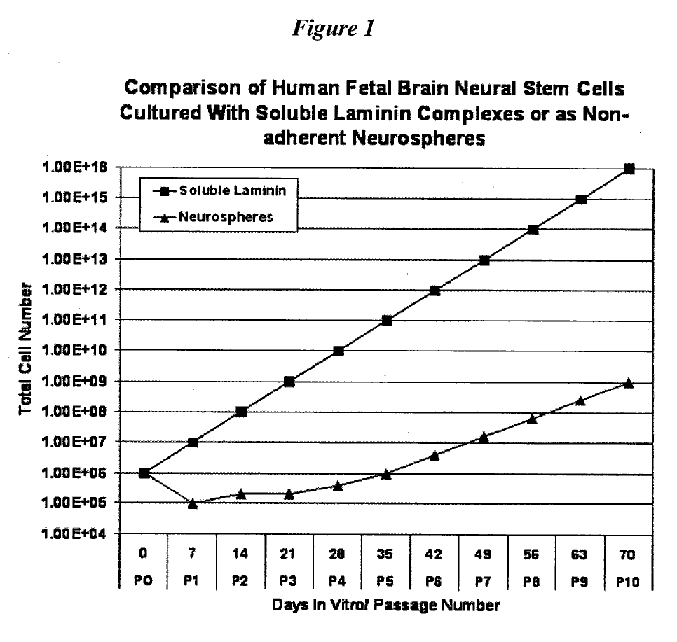

The soluble laminin with associated sLAF(s) (from Sigma, Cat. # L2020) were added at 1 μg/ml to one flask, and both flasks were cultured for 7 days in a 5% CO₂, humidified incubator. The flask without added soluble laminin formed neurospheres, while the flask containing soluble laminin and associated sLAF(s) grew as adherent cells. After each 7 day passage, cells from both culture types were trypsinized into single-cell suspensions, counted in a hemacytometer and expanded similarly into another T-150. The calculated total cell expansion potential was then expressed over time and graphed in FIG. 1.

### Example 2

Timed pregnant Fisher rats were obtained from Simonsen Labs, and were euthanized with CO₂ gas. The E18 embryos were dissected, and the entire brain cavity was removed, minced with scissors, then digested in 1 mg/ml papain in DMEM/F12 medium at 37C in a water bath for 30 minutes. After trituration with a 5 ml pipet, the cell suspension was diluted with neural stem cell medium (see FIG. 1 above), filtered through a 70 μM cell strainer, then centrifuged at 200 g for 5 minutes.

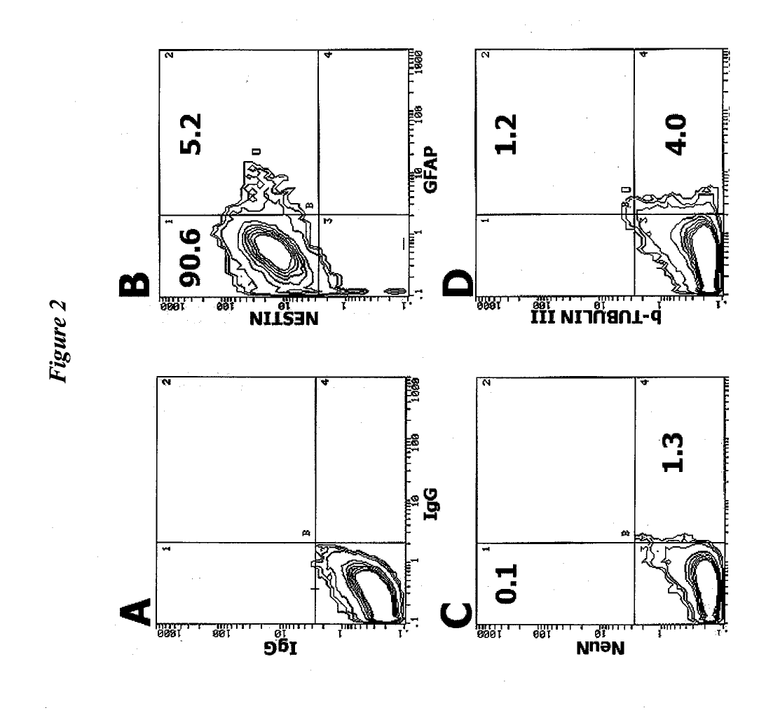

The cell pellet was resuspended in neural stem cell medium at 2 ml per embryo, and each 2 ml aliquot was cultured in a 150 cm² T-flask in neural stem cell medium with 1 μg/ml soluble laminin and associated sLAF(s), 10 ng/ml bFGF, and 5 ng/ml LIF in a 5% CO₂, humidified incubator. After 7 days cultures were trypsinized into single cell suspensions and 1.5×10⁶ cells were subcultured as described above. After 4 passages the resulting single cell suspension was processed for intracellular FACS using a commercial kit (Orion BioSolutions, IntraCyte-rNSC, Cat.#01026) according to the manufacturer's instructions. After staining, cells were analyzed on a Coulter Epics ELITE cytometer and approximately 20,000 cells were collected for analysis with each antibody. Intact cells were identified by characteristic forward and side light-scatter patterns(about 80% of events) and dual-parameter contour plots are shown in FIG. 2.

The percentage of cells immunopositive above the negative control is shown. Note that 95.8% of cells are nestin immunoreactive, while only 0.1% of cells are NeuN positive, 1.2% of cells are beta-tubulin III immunoreactive, 1.3% of cells are Myelin Basic Protein immunoreactive, and 5.2% of cells are GFAP immunoreactive.

All references cited herein are hereby incorporated by reference in their entireties, whether previously specifically incorporated or not. As used herein, the terms “a”, “an”, and “any” are each intended to include both the singular and plural forms.

Having now fully described this invention, it will be appreciated by those skilled in the art that the same can be performed within a wide range of equivalent parameters, concentrations, and conditions without departing from the spirit and scope of the invention and without undue experimentation. While this invention has been described in connection with specific embodiments thereof, it will be understood that it is capable of further modifications. This application is intended to cover any variations, uses, or adaptations of the invention following, in general, the principles of the invention and including such departures from the present disclosure as come within known or customary practice within the art to which the invention pertains and as may be applied to the essential features hereinbefore set forth.

What is claimed is:

1. A composition comprising a cell of the central nervous system (CNS) that has been cultured in the presence of a solution comprising laminin and nidogen.

2. The composition of claim 1 wherein said cell is not terminally differentiated and/or not postmitotic; or
wherein said cell is selected from a neural stem cell, a neural progenitor cell, a motor neuron progenitor, an oligodendroglial progenitor cell, and any CNS-derived cell which plays a beneficial role in the regenerative response to CNS damage, inflammation, or infection; or
wherein said cell is a nestin expressing neuroepithelial cell or radial glial cell-like neuroglial progenitor cell.

3. The composition of claim 1 wherein said cell is a descendant of a primate-derived primordial germ cell, or a human embryonic stem cell.

4. The composition of claim 1 wherein said solution further comprises a heparin sulfate proteoglycan; collagen type IV; secreted protein, acidic, rich in cysteine (SPARC); tenascin; reelin; thrombospondin; or a combination of any number of the foregoing.

5. A cell of the CNS that has been cultured in the presence of a solution comprising laminin and nidogen.

6. The cell of claim 5 wherein said cell is not terminally differentiated and/or not postmitotic; or
wherein said cell is selected from a neural stem cell, a neural progenitor cell, a motor neuron progenitor, an oligodendroglial progenitor cell, and any CNS-derived cell which plays a beneficial role in the regenerative response to CNS damage, inflammation, or infection; or
wherein said cell is a nestin expressing neuroepithelial cell or radial glial cell-like neuroglial progenitor cell.

7. The cell of claim 5 wherein said cell is a descendant of a primate-derived primordial germ cell, or a human embryonic stem cell.

8. The cell of claim 5 wherein said solution further comprises a heparin sulfate proteoglycan; collagen type IV; secreted protein, acidic, rich in cysteine (SPARC); tenascin; reelin; thrombospondin; or a combination of any number of the foregoing.

9. A cell that is derived from the cell of claim 5.

10. The cell of claim 9 wherein said cell displays a decreased level of nestin expression, optionally with an increased level of TUJ1/β-tubulin expression; a increased level of glial fibrillary acidic protein (GFAP) expression; or an increase in expression of at least one marker of a differentiated CNS cell type.

11. The cell of claim 10 wherein said marker is selected from acetyl cholinesterase; vesicular acetylcholinesterase; gamma-aminobutyric acid (GABA); serotonin;
a synapse marker, including synaptophysin and synaptogamin; post-synaptic density protein 95 (PSD-95); myelin basic protein; myelin associated glycoprotein (MAG); proteo-lipid protein (plp or DM20); NG2 proteoglycan; CD24; CD133; CD49f; tyrosine hydroxylase; and L-3,4-dihydroxyphenylalanine (DOPA) decarboxylase.

12. The cell of claim 9 wherein said cell is an astrocyte, a neuron, a microglial cell, an oligodendrocyte, or an oligodendroglial cell.

13. A daughter or descendant cell obtained by passage of the cell of claim 5.

14. A method of culturing a cell of the CNS, said method comprising
culturing said cell in a medium containing soluble laminin and soluble nidogen.

15. The method of claim 14 wherein said laminin and nidogen are in a soluble complex in said medium.

16. The method of claim 14 wherein said medium comprises FGF-2.

17. The method of claim 14 wherein said medium comprises LIF (leukemia inhibitory factor).

18. The method of claim 14 wherein said culturing is in a three dimensional format.

19. A method of determining the effects of a candidate agent on a cell of the CNS differentiation, growth, viability, or metabolic activity, said method comprising
culturing said cell in a medium containing soluble laminin and soluble nidogen;
altering the culture conditions for said cell to permit differentiation, such as by growth factor withdrawal, after said cell has been contacted with said candidate agent; and
observing the effects of said candidate agent on said differentiation in comparison to another cell cultured under the same conditions to permit differentiation in the absence of said candidate agent.

20. The method of claim 19 wherein said culture conditions to permit differentiation are selected from withdrawal of soluble laminin and nidogen and/or induction with retinoic acid, or a neurotrophic peptide factor such as neural growth factor (NGF), neuregulins, glial cell derived neurotrophic factor (GDNF), ciliary neurotrophic factor (CNTF), and brain derived neurotrophic factor (BDNF).
